(19) United States
(12) Patent Application Publication
(10) Pub. No.: US 2005/0143735 A1
(43) Pub. Date: Jun. 30, 2005

(54) Double compression unloadable screw system

(76) Inventor: Richard F. Kyle, Long Lake, MN (US)

Correspondence Address: STEVEN J. KEOUGH, 1912 SUMMIT AVE, ST PAUL, MN, 55105, US

(21) Appl. No.: 10/836,020

(22) Filed: Apr. 29, 2004

Related U.S. Application Data
(60) Provisional application No. 60/466,498, filed on Apr. 29, 2003.

(51) Int. Cl. A61F 2/30

(52) U.S. Cl. 606/60

(57) ABSTRACT

A bone screw assembly for joining bone fragments having a screw shaft, a compression head and a sleeve. The compression head has external threads for providing double compression of the bone fragments. The sleeve extends along and around the screw shaft to provide reinforcing strength and alignment to the screw.

### PRIORITY

This application claims priority to Provisional Application No. 60/466,498 filed Apr. 29, 2003.

### FIELD OF THE INVENTION

The invention generally relates to an apparatus and method of bone surgery. In particular, this invention is for uniting bone fragments, such as in a fractured hip.

### BACKGROUND OF THE INVENTION

Hip fractures commonly occur through the neck of the femur and less frequently through the femoral head. Surgical repair of such fractures involves insertion of a screw from the lateral proximal femur, through the neck of the femur and into the femoral head. This results in uniting of the bone fragments under a certain amount of compression and allows for bone healing. These screws commonly have threads at the proximal and distal ends and have a central portion with no threads. The result is that the distal threads engage the most internal bone to be joined and the proximal threads engage the most proximal portion of bone to be joined, pulling together the bone fragments across the fracture line. This results in a certain degree of pressure across the fracture which promotes healing of the bone. However, it is desirable to increase the amount of pressure across the fractured bone fragments to stabilize the bones and improve bone healing. Furthermore, after the patient returns to weight bearing activities, these hip screws are placed under stress. The forces acting upon the hip screws can cause them to fail in a predictable region along their lengths. There is therefore a need to improve hip screws to avoid these predictable failures.

One known method of providing increased compression to bone fragments involves the use of a bone screw with threads at the proximal and distal ends and a separate head. After insertion of the screw, the head is threaded onto the proximal end of the screw to provide additional compression to the bone fragments.

### SUMMARY OF THE INVENTION

The present invention provides an apparatus and method for both improving the compression of the bone fragments as well as strengthening of the bone screw. It includes a screw shaft with threads on the proximal and distal ends to unite the bone fragments, a head which threads over the proximal end of the screw shaft and provides increased compression on the bone fragments and a sleeve which slides or threads around the bone screw and provides reinforcing strength. The compression head and the strengthening sleeve may be a single unit or may be two distinct components. Therefore, the invention has the advantage that the combined head and sleeve, or the head and sleeve individually, can be removed in the future after initial insertion of the assembly.

The compression head and the sleeve serve to increase the compression pressure on the bone fragments and to decrease the likelihood of screw failure. After the screw shaft is inserted through the bone fragments to unite them under pressure, the reinforcing sleeve (or head and sleeve combination) slides or threads around the proximal end of the inserted screw shaft. It is of a length sufficient to extend across the region of the screw shaft that is under stress and prone to failure. The sleeve thus provides reinforcing strength to this area of the bone screw to decrease the likelihood of future bone screw breakage. The compression head (if not part of a head and sleeve combination) is then threaded onto the proximal threaded end of the bone screw. In this way, the head (or the head portion of the combined head and sleeve) creates further compression across the fracture line such that there is a double compression of the bone fragments, which is desirable for bone healing.

The removable nature of the compression head and reinforcing sleeve can serve various purposes. For example, the sleeve may be made of a material which reveals signs of stress when imaged, such as by an x-ray. When a stressed sleeve is thus detected, it can then be removed and replaced by a new sleeve. The new sleeve then continues to provide reinforcement to the bone screw and further decreases the chance of bone screw failure. Such a sleeve therefore provides protection against bone screw failure in multiple ways. The sleeve provides physical reinforcement to the screw when initially installed. It also prevents bone screw failures by revealing, through imaging or other manner, the need for replacement of the stressed sleeve with a new sleeve.

The system can also be used to provide bone dynamization to promote bone healing. After insertion of the bone screw assembly and a period of bone regrowth under static conditions, either the compression head, the sleeve or both may be moved or removed. Removal of these elements decreases the compression on the bone fragments and allows some increased mobility of the bone fragments. This change in conditions may be used to stimulate continued bone re-growth and strengthening after initial re-growth has occurred.

### BRIEF DESCRIPTION OF THE DRAWINGS

FIG. 1 is a sectional view showing a bone screw assembly with a compression unit and a screw shaft.

FIG. 2 is an exploded sectional view showing a bone screw assembly with a compression unit and a screw shaft.

FIG. 3 is an exploded perspective view of a bone screw assembly with a compression unit and a screw shaft.

FIG. 4 is an exploded sectional view showing a bone screw assembly with a compression head, a sleeve, and a screw shaft.

FIG. 5 is an exploded sectional view showing a bone screw assembly with a compression head, a sleeve, and a screw shaft.

FIG. 6 is a sectional view showing a fractured femoral neck with the bone screw of FIG. 1 joining the bone fragments.

FIG. 7 is a sectional view showing a fractured femoral neck with the bone screw and compression unit of FIG. 1 joining the bone fragments.

### DETAILED DESCRIPTION OF THE PREFERRED EMBODIMENTS

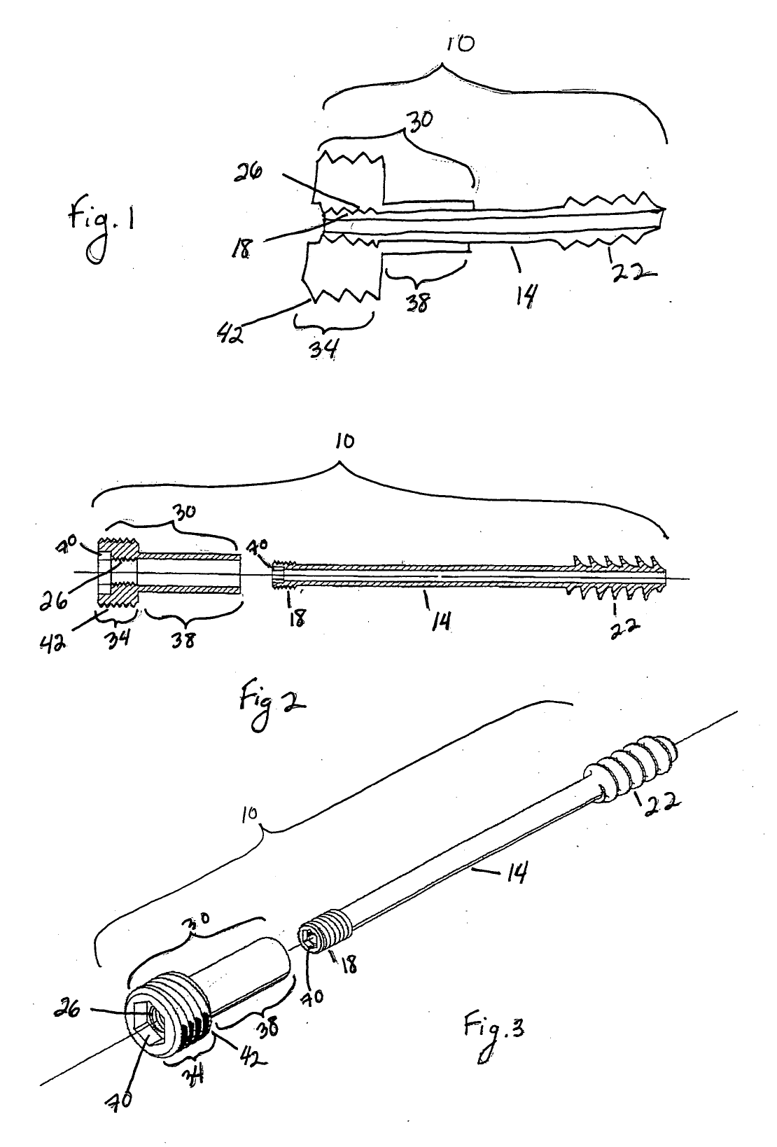

The bone screw assembly of the present invention is shown in FIGS. 1 through 7. As shown in FIGS. 1 through 3, the bone screw assembly 10 includes a screw shaft 14 having an elongate wall structure and external threads 18, 22 at the proximal and distal ends, respectively. The distal end of the screw shaft is shaped to allow it to penetrate the bone. The screw shaft 14 may be partially or fully canulated or may be solid. The proximal screw threads 18 are shaped to engage the internal threads 26 of the compression unit 30. The distal screw threads 22 are shaped to engage the distal bone fragments and may comprise any of a plurality of functional patterns/shapes.

The compression unit 30 is shaped to advance along and around a portion of the screw shaft 14. The compression unit 30 includes a compression unit head 34 and a compression unit sleeve 38. The proximal first portion of the compression unit 30 is the compression unit head 34. The compression unit head 34 includes internal threads 26 for connecting with the proximal threads 18 of the screw shaft 14 and external threads 42 for providing the double compression bone-engagement function. The distal second portion of the compression unit 30 is the compression unit sleeve 38. The compression unit sleeve 38 may slide along a portion of the screw shaft 14 such that it is in contact with the screw shaft 14 or there may be a gap between the screw shaft 14 and the compression unit sleeve 38. The central portion of the screw shaft 14 may be without external threads, may have external threads running its entire length or may have one or more segments of external threads.

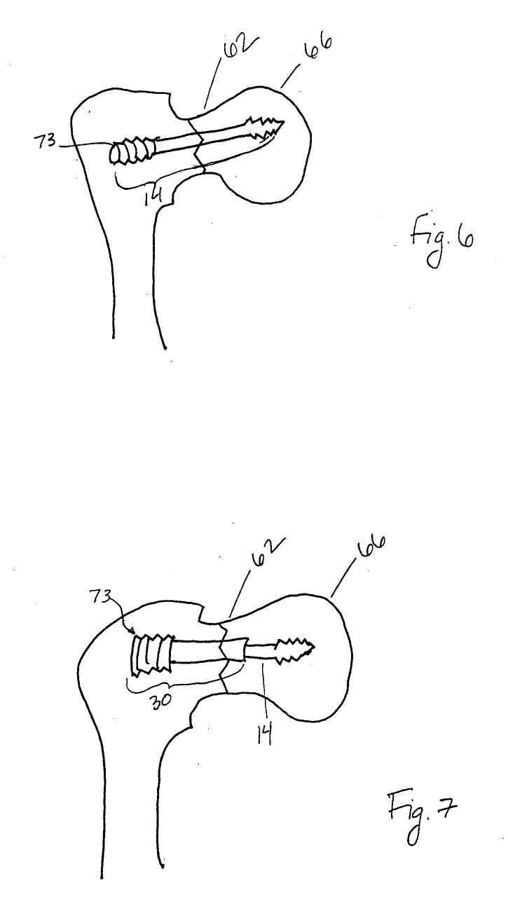

Alternatively, as shown in FIGS. 4 and 5, the compression head 46 and sleeve 50 may be separate components of the bone screw assembly, as shown in FIGS. 4 and 5, rather than combined as a single unit. In this alternative, the compression head 46 contains internal threads 26 and external threads 42. The sleeve 50 contains internal threads 54 and may or may not contain external threads 58. When the sleeve has external threads 58, as shown in FIG. 4, these threads are shaped to connect with the internal threads 26 of the compression head 42, which advances around the sleeve 50. The internal threads 58 of the sleeve 50 are shaped to connect with the proximal threads 18 of the screw shaft 14. When the sleeve 50 is without external threads, as shown in FIG. 5, the sleeve 50 advances along the screw shaft 14 with its internal threads 54 engaging the proximal threads 18 of the screw shaft 14. The compression head 46 then follows, after the sleeve 50, advancing around the proximal end of the screw shaft 14 in a location proximal to the sleeve 50 such that the internal threads 26 of the compression head 46 engage with the proximal threads 18 of the screw shaft 14.

As shown in FIG. 6, the screw shaft 14 is inserted through the neck 62 of the femur and into the head 66 of the femur. After insertion of the screw shaft 14, the compression unit 30, as shown in FIG. 7, or the separate sleeve 50 and compression head 46, are advanced along and around the screw shaft 14 to provide double compression of the bone fragments and strengthening of the screw shaft 14. Such strengthening may be quite important in view of the stresses on the screw shaft generally at between about 20%-35% of the length of the screw shaft as measured from the proximal end 73.

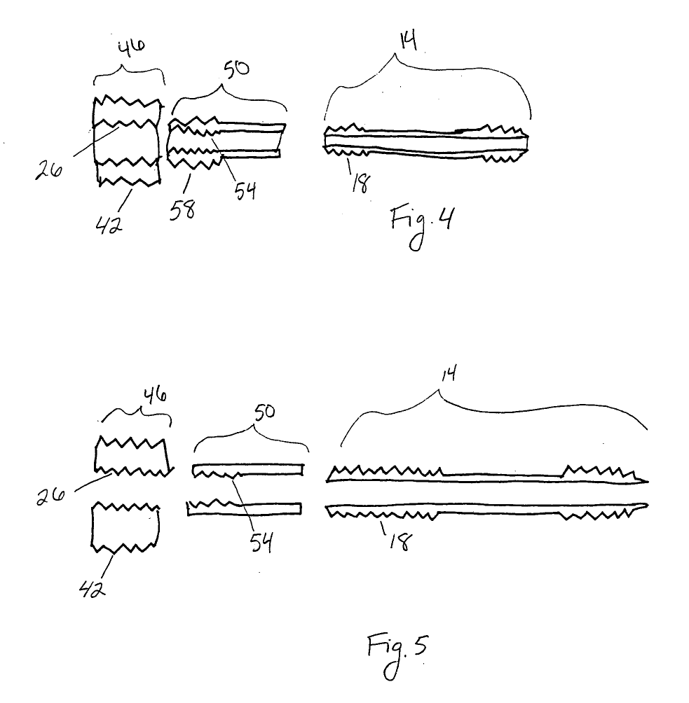

The proximal ends of both the screw shaft 14 and the compression unit 30 may be shaped to allow selective engagement with one or more insertion devices or drivers, as shown in FIGS. 2 and 3. For example, these components may have insets 70, such as hexagonal insets, or other features to allow an insertion device with a hexagonally shaped tip or other type of insertion device to drive the elements into the bone.

In order to prevent the compression unit 30, the sleeve 50 or the compression head 46 from advancing too far distally on the screw shaft, the invention may include structure for stopping advancement of these elements at a desired point. For example, the proximal end of the compression unit 30 may include a rim which would abut against the proximal end of the screw shaft after the compression unit is completely advanced onto the screw shaft. Alternatively, the proximal threads 18 of the screw shaft 14 could include a closed end to the threading such that the advancing internal threads 26 of the compression unit would be stopped at that point during the threading process. Another alternative would be an external projection on the screw shaft such as a nub or a ring around the screw shaft which would block the distal advancement of the compression unit 30 or the sleeve 50 at a predetermined point.

The invention may also include the use of a coating or coatings between the screw shaft and the compression unit sleeve. This coating could be supplied on the screw shaft 14 or in the lumen of the sleeve 50 or of the compression unit sleeve 38. The coating could serve to facilitate sliding of the sleeve 50, to prevent bony in-growth between the sleeve 50 and the screw shaft 14, to do both, or to serve any other biomedical device-related function, according to need. In addition, the screw shaft 14 could include apertures through which material could be delivered to the surrounding tissue or which could be used to allow tissue in-growth.

The compression unit 30, the compression head 46 and the sleeve 50 have the advantageous option of being removable and replaceable. For example, they could be removably connected to the screw shaft at the time of insertion of the bone screw assembly. Later, they might be removed from the screw shaft in order to facilitate dynamization. In addition, the sleeve 50 or the compression unit sleeve 38 could be made of a material that indicates stress points when imaged radiologically or with other medical imaging techniques, including, for example, X-ray, ultrasound, or others. When imaging reveals stress in the reinforcing sleeve, the compression unit 30 or the sleeve 50 could be adjusted, removed or replaced. However, when it is not desirable for the compression unit 30 or the sleeve 50 and head 46 to be removable and replaceable, they could be permanently connected to the screw shaft 14 after installation. It is further recognized that the pitch, height and other features of the threads referred to herein may be modified to achieve single compression ratios for low values, double compression ratios for high value, or may comprise more standard or universal ratios. The sleeves 50 are a unique combination when used for strengthening, imaging for stress, or for implementing the guiding force to prevent retrograde and lateral motion of the screw shaft or attached components after implant. For example, in one embodiment, initial compression is achieved by placing at least one screw shaft into a patient across a fracture site. This achieves initial compression which is then further assisted by use of the compression unit and sleeve. The bones then stick together and commence the healing process. However, it is often essential to subsequently release the compression forces of the compression unit and allow the natural muscle force of the patient to further accelerate healing at the site. During this process the sleeve maintains natural compression and guiding alignment of the screw shaft to prevent undesired migration. This is particularly advantageous when more than one screw is inserted, which is common, to prevent interference or disturbance by one screw against another. The invention enables single then double compression, followed by aligned natural fracture impaction, while serving as an anchor or backstop to prevent retrograde motion of the screw shaft. The invention may utilize or enable various combinations of the above features and results, and may include components having bio-resorbable characteristics.

1. A bone screw assembly for use in joining bone fragments, comprising:
a screw shaft having an elongate wall structure and having external threads at a distal end and external threads at a proximal end; and
a compression unit shaped to advance along and around a portion of said screw shaft, the unit having a proximal first portion with external threads and internal threads to form a head and a distal second portion extending distally from said first portion to form a screw shaft strengthening sleeve.

2. The bone screw assembly of claim 1, wherein the screw shaft elongate wall structure forms an inner lumen extending through a portion of the screw shaft.

3. The bone screw assembly of claim 1, wherein the screw shaft elongate wall structure forms an inner lumen extending the entire length of the screw shaft.

4. The bone screw assembly of claim 1, wherein the screw shaft includes an external central portion with no threads between the proximal and distal threaded ends of the screw shaft.

5. The bone screw assembly of claim 1, wherein the screw shaft has external threads between the proximal and distal threaded ends of the screw shaft.

6. The bone screw assembly of claim 1, wherein the proximal end of the screw shaft is shaped to allow selective engagement with an insertion device.

7. The bone screw assembly of claim 1, wherein the proximal end of the compression unit is shaped to allow selective engagement with an insertion device.

8. The bone screw assembly of claim 1, wherein the head portion of the compression unit is shaped to thread along the proximal threaded end of the screw shaft.

9. The bone screw assembly of claim 1, comprising structure for preventing the compression unit from advancing distally on the screw shaft beyond a predetermined point.

10. The bone screw assembly of claim 1, further comprising a coating between the screw shaft and the compression unit sleeve to facilitate sliding.

11. The bone screw assembly of claim 1, further comprising a coating between the screw shaft and the compression unit sleeve to prevent bony or other tissue ingrowth.

12. The bone screw assembly of claim 1, wherein the compression unit sleeve is comprised of a material that indicates stress points when imaged.

13. The bone screw assembly of claim 12, wherein a compression unit with a stressed sleeve is removable and replaceable.

14. The bone screw assembly of claim 1, wherein the screw shaft is in contact with the compression unit sleeve.

15. The bone screw assembly of claim 1, wherein there is a gap between the screw shaft and the compression unit sleeve.

16. The bone screw assembly of claim 1, wherein the screw shaft comprises wall features forming apertures.

17. The bone screw assembly of claim 1, wherein the compression unit is permanently connected to the screw shaft after installation.

18. The bone screw assembly of claim 1, wherein the compression unit is removably connected to the screw shaft.

19. The bone screw assembly of claim 18, wherein the compression unit is removable to facilitate dynamization.

20. A bone screw assembly for use in joining bone fragments, comprising:
a screw shaft having an elongate wall structure and having external threads at the distal end and external threads at the proximal end; and
a sleeve shaped to advance along and around a portion of said screw shaft and having a proximal portion with internal threads for engaging said screw shaft external threads; and
a compression head shaped to advance along and around said screw shaft and having internal threads and external threads.

21. The bone screw assembly of claim 20, wherein the sleeve and compression head are may be loosened or removed to facilitate dynamization.

22. A bone screw assembly for use in joining bone fragments, comprising:
a screw shaft having an elongate wall structure and having external threads at the distal end and external threads at the proximal end; and
a sleeve shaped to advance along and around a portion of said screw shaft and having a proximal portion with external threads and internal threads for engaging said screw shaft external threads; and
a compression head shaped to advance along and around a portion of said sleeve and said screw shaft and having internal threads and external threads.

23. A method of providing a double compression strengthened bone screw assembly to join bone fragments in a host, comprising the steps of:
selecting a site for placement of a bone screw assembly;
inserting a bone screw shaft into the selected location, said bone screw shaft comprising:
a screw shaft having an elongate wall structure and having external threads at the distal end and external threads at the proximal end; and
advancing an alignment and strengthening sleeve and a compression head distally along and around said bone screw shaft to provide a double compression strengthened bone screw assembly.

24. A method of detecting bone screw stress in a host, comprising the steps of:
selecting a site for placement of a bone screw assembly;
inserting a bone screw shaft into the selected location, said bone screw shaft comprising:
a screw shaft having an elongate wall structure and having external threads at the distal end and external threads at the proximal end;
advancing an alignment sleeve and compression head distally along and around said bone screw shaft, wherein said sleeve is comprised of a material that indicates stress points when imaged; and
imaging said sleeve to detect stress points.
